(19) United States
(12) Patent Application Publication
(10) Pub. No.: US 2026/0061090 A1
(43) Pub. Date: Mar. 5, 2026

(54) MEDICAL TISSUE ADHESIVE FOR PANCREATIC PLUGGING AND PREPARATION METHOD AND USE THEREOF

(71) Applicant: Beijing Biosis Healing Biological Technology Co., Ltd., Daxing District, Beijing (CN)

(72) Inventors: Xueqiao YU, Daxing District, Beijing (CN); Yiqian HUANG, Daxing District, Beijing (CN); Pengfei WEI, Daxing District, Beijing (CN); Wei JING, Daxing District, Beijing (CN); Bo ZHAO, Daxing District, Beijing (CN)

(21) Appl. No.: 18/855,062

(22) PCT Filed: Apr. 12, 2023
(86) PCT No.: PCT/CN2023/087902

§ 371 Date: May 15, 2025

(30) Foreign Application Priority Data
Apr. 14, 2022 (CN) 202210395320.6

(51) Int. Cl. A61L 24/04; A61L 24/00

(52) U.S. Cl. CPC A61L 24/046; A61L 24/0015; A61L 2430/36

(57) ABSTRACT

The present disclosure relates to a medical tissue adhesive for pancreatic plugging and the preparation method and use thereof. The medical tissue adhesive composition forming the medical tissue adhesive of the present disclosure comprises a medical tissue adhesive component and a chelating agent component; wherein the medical tissue adhesive component comprises a first component and a second component, the first component comprises four-arm polyethylene glycol amine, and the second component comprises four-arm-polyethylene glycol-succinimidyl glutarate; wherein m and n are natural numbers; and the chelating agent component comprises polyphenol. The medical tissue adhesive prepared from the medical tissue adhesive composition of the present disclosure is capable of firmly binding to the surface of the pancreatic tissue, and is capable of withstanding the dynamic pressure as a result of pancreatic juice leakage and the enzymatic degradation activity of pancreatic juice. More importantly, the medical tissue adhesive of the present disclosure has an excellent antibacterial ability and is capable of preventing the infection caused by the long-term accumulation of the leaked pancreatic juice.

### TECHNICAL FIELD

The present disclosure belongs to the field of biomedical materials, and specifically relates to a medical tissue adhesive composition for pancreatic plugging, a medical tissue adhesive and a preparation method and use thereof, and a kit for preparing medical tissue adhesive.

### BACKGROUND

Medical tissue adhesive is a tissue sealing material used for sealing and plugging in the case of tissue trauma, bleeding, or fluid (tissue fluid or gas) leakage. At present, the mainstream medical tissue adhesives may be divided into four categories: bio-stable type, biodegradable type, synthetic stable type, and synthetic degradable type.

Bio-stable medical tissue adhesive is a material obtained by crosslinking a bio-based raw material with the aid of a chemical reagent, e.g., Cryolife's BioGlue, which is a tissue sealing material having an albumin derived from a biological source and glutaraldehyde as main components. Biodegradable medical tissue adhesive is a biodegradable and absorbable material composed fully of a bio-based raw material, e.g., Baxter's Tisseel, which is a fibrin-derived biodegradable tissue glue. Synthetic stable medical tissue adhesive is a material resulting from the self-polymerization of a polymer (primarily α-cyanoacrylate) which undergoes anionic polymerization at the tissue interface to form tissue bonding, but its polymer is not biodegradable. Synthetic degradable medical tissue adhesive is mainly chemically-modified polyethylene glycol, and polyethylene glycol would be metabolized and excreted out of the body through cell membrane penetration and endocytosis as the crosslink bond breaks.

Typically, the bio-stable medical tissue adhesive, such as BioGlue, uses glutaraldehyde as a molecule for crosslinking albumin and the residual glutaraldehyde has high cytotoxicity, BioGlue is therefore easy to cause an inflammatory reaction when implanted into the body, and furthermore, the crosslink bond formed between glutaraldehyde and albumin is not biodegradable. The biodegradable medical tissue adhesive such as Tisseel forms a gel in situ mainly by forming crosslink via fibrin and thrombin (factor), calcium chloride, etc. By activating the coagulation cascade reaction based on fibrin and thrombin (factor), this product is mainly used for hemostasis, while the tissue adhesiveness is relatively weak. For the synthetic stable medical tissue adhesive such as Histoacryl, n-butyl 2-cyanoacrylate undergoes anionic polymerization to from poly(n-butyl α-cyanoacrylate), and this polymer macromolecule is not biodegradable; furthermore, monomers unreacted during the polymerization has certain biotoxicity. The synthetic degradable material, such as Coseal, is composed of four-arm polyethylene glycol ether tetrasuccinimide glutaric acid and four-arm polyethylene glycol ether tetramercaptan, and a medical tissue adhesive with tissue adhesiveness could be formed in situ by mixing these two components, but it is currently not suitable for the plugging of pancreatic juice leakage.

Within the compliant range of application, there is currently no medical tissue adhesive suitable for the plugging of pancreatic juice leakage. On the one hand, due to the sensitivity of the pancreatic tissue, common tissue binders such as α-cyanoacrylate release heat upon polymerization, which damages the pancreatic tissue, or the potential biotoxicity of BioGlue endangers the function and metabolism of the pancreatic tissue and cells. On the other hand, long-term leakage and accumulation of pancreatic juice are easy to cause infection and damage, and medical tissue adhesives do not have an antibacterial function.

Therefore, it becomes an urgent technical problem to develop a medical tissue adhesive that is usable for the plugging of pancreatic juice, has an excellent antibacterial ability, and is capable of preventing the infection caused by the long-term accumulation of the leaked pancreatic juice.

### SUMMARY

### Problems to be Solved by the Invention

In view of the technical problems existing in the prior art, the present disclosure provides a medical tissue adhesive composition and a medical tissue adhesive, and this medical tissue adhesive is capable of firmly binding to the surface of the pancreatic tissue and is capable of withstanding the dynamic pressure as a result of pancreatic juice leakage and the enzymatic degradation activity of pancreatic juice. More importantly, the medical tissue adhesive of the present disclosure has an excellent antibacterial ability and is capable of preventing the infection caused by the long-term accumulation of the leaked pancreatic juice.

Furthermore, the present disclosure further provides a preparation method of a medical tissue adhesive. This preparation method is simple and easy to perform with easy access to raw materials and relatively low costs.

### Solutions to Problems

In the first place, the present disclosure provides a medical tissue adhesive composition for preparing a plugging article for pancreatic juice leakage, comprising a medical tissue adhesive component and a chelating agent component, wherein
the medical tissue adhesive component comprises a first component and a second component, the first component comprises four-arm polyethylene glycol amine, and the second component comprises four-arm-polyethylene glycol-succinimidyl glutarate, wherein
the four-arm polyethylene glycol amine has a structural formula as represented by formula (I):

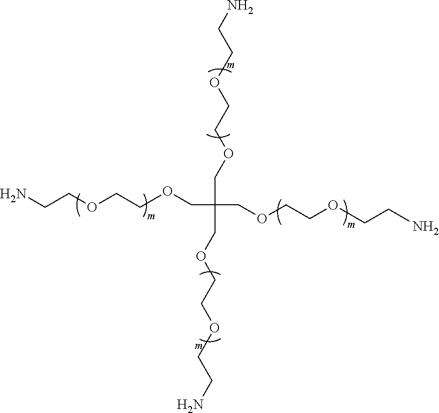

the four-arm-polyethylene glycol-succinimidyl glutarate has a structural formula as represented by formula (II):

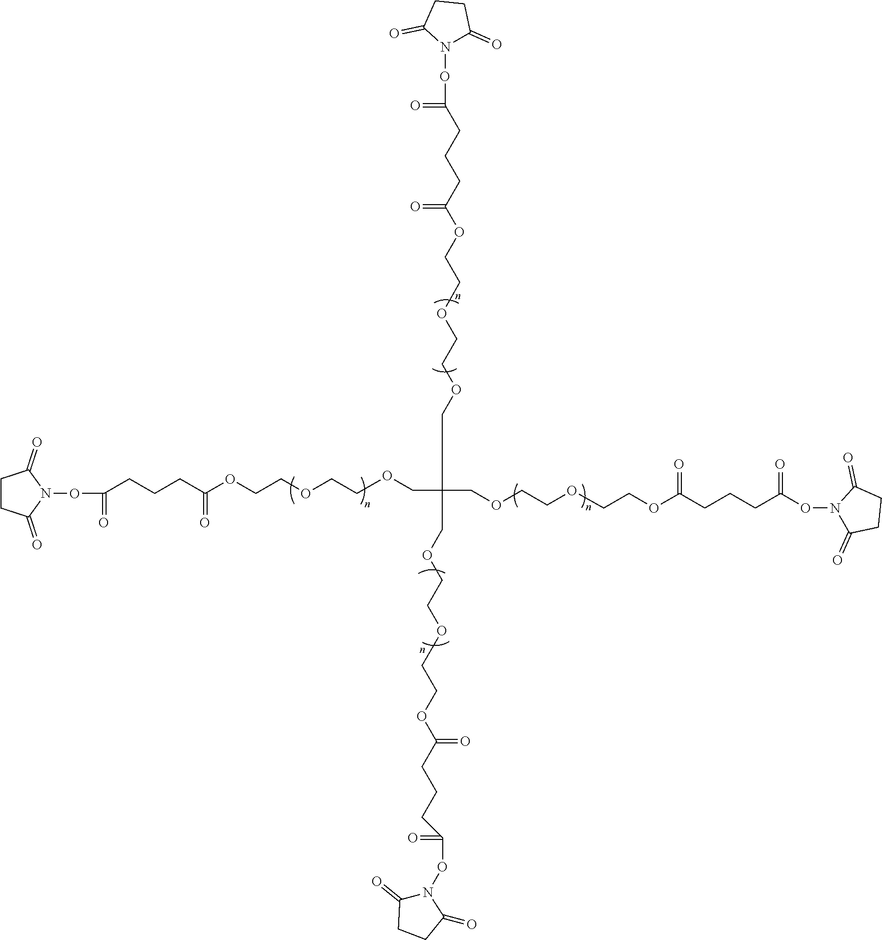

wherein m and n are natural numbers; and
the chelating agent component comprises polyphenol.

In some embodiments, in the medical tissue adhesive composition according to the present disclosure, the four-arm-polyethylene glycol-succinimidyl glutarate has a weight-average molecular weight of 2000 to 20000 Da, preferably 5000 to 15000 Da, more preferably 8000 to 12000 Da; and the four-arm polyethylene glycol amine has a weight-average molecular weight of 2000 to 20000 Da, preferably 5000 to 15000 Da, more preferably 8000 to 12000 Da.

In some embodiments, in the medical tissue adhesive composition according to the present disclosure, the polyphenol comprises tannic acid, catechol, pyrogallol or resveratrol, or a combination of two or more selected from these components, preferably comprises tannic acid.

In some embodiments, in the medical tissue adhesive composition according to the present disclosure, the mass ratio of the four-arm-polyethylene glycol-succinimidyl glutarate to the chelating agent is 1:(0.003 to 0.03), preferably 1:(0.01 to 0.02); and/or
the mass ratio of the first component to the second component is 1:(0.8 to 1.2), preferably 1:(0.9 to 1.1), more preferably 1:(0.95 to 1.05).

In some embodiments, in the medical tissue adhesive composition according to the present disclosure, the medical tissue adhesive composition further comprises one or a combination of two or more selected from a metal compound, a polypeptide, and an antimicrobial drug; the metal compound comprises one or a combination of two or more selected from magnesium oxide, zinc oxide, and zinc carbonate, and the antimicrobial drug comprises one or a combination of two or more selected from vancomycin, gentamicin, fusidine, and a quaternary ammonium salt.

The present disclosure further provides a medical tissue adhesive for preparing a plugging article for pancreatic juice leakage, wherein the medical tissue adhesive is formed via the reaction of the first component and the second component of the medical tissue adhesive composition according to the present disclosure in the presence of a chelating agent component.

In some embodiments, in the medical tissue adhesive according to the present disclosure, the medical tissue adhesive has at least one of the following characteristics:
the medical tissue adhesive having a tissue adhesive strength of 2 to 50 kPa;
the medical tissue adhesive having a degradation cycle of 30 to 60 days; and
the medical tissue adhesive having a bacteriostatic rate of 95% or more.

The present disclosure further provides a preparation method of the medical tissue adhesive according to the present disclosure, comprising the steps of:
preparing a solution A comprising a first component and a chelating agent;
preparing a solution B comprising a second component; and
mixing the solution A and the solution B to obtain the medical tissue adhesive.

In some embodiments, in the preparation method according to the present disclosure, the step of preparing the solution A comprising four-arm polyethylene glycol amine and the chelating agent comprises:
preparing a solution containing four-arm polyethylene glycol amine and a solution containing the chelating agent respectively, and mixing two solutions to obtain the solution A; or
preparing a solution containing four-arm polyethylene glycol amine, and adding the chelating agent to the solution containing the four-arm polyethylene glycol amine to obtain the solution A; or
preparing a solution containing the chelating agent, and adding the four-arm polyethylene glycol amine to the solution containing the chelating agent to obtain the solution A; or
mixing the four-arm polyethylene glycol amine and the chelating agent, and adding the mixture to a solvent to obtain the solution A.

In some embodiments, in the preparation method according to the present disclosure, in the solution A, the chelating agent component has a mass percentage concentration of 0.06 to 0.6% (m/v), preferably 0.1 to 0.50% (m/v), more preferably 0.2 to 0.4% (m/v), further preferably 0.30% (m/v); and/or
in the solution A, the four-arm polyethylene glycol amine has a mass percentage concentration of 10 to 25% (m/v), preferably 18 to 22% (m/v).

In some embodiments, in the preparation method according to the present disclosure, in the solution B, the four-arm-polyethylene glycol-succinimidyl glutarate has a mass percentage concentration of 10 to 25% (m/v), preferably 18 to 22% (m/v).

The present disclosure further provides a medical tissue adhesive kit, comprising the medical tissue adhesive composition according to the present disclosure; preferably, the medical tissue adhesive component and the chelating agent component of the medical tissue adhesive composition are stored separately; more preferably, the first component and the second component of the medical tissue adhesive composition are stored separately.

The present disclosure further provides the use of the medical tissue adhesive according to the present disclosure or a medical tissue adhesive prepared and obtained by the preparation method of the medical tissue adhesive according to the present disclosure in the preparation of a plugging article for pancreatic juice leakage.

### Effects of Invention

The medical tissue adhesive prepared from the medical tissue adhesive composition of the present disclosure is capable of firmly binding to the surface of the pancreatic tissue, and is capable of withstanding the dynamic pressure as a result of pancreatic juice leakage and the enzymatic degradation activity of pancreatic juice. More importantly, the medical tissue adhesive of the present disclosure has an excellent antibacterial ability and is capable of preventing the infection caused by the long-term accumulation of the leaked pancreatic juice.

Furthermore, the preparation method of the medical tissue adhesive of the present disclosure is simple and easy to perform with easy access to raw materials and relatively low costs, thereby being suitable for mass production.

### BRIEF DESCRIPTION OF THE DRAWINGS

FIG. 1 shows a schematic diagram of the gelation mechanism of the medical tissue adhesive of the present disclosure.

FIG. 2 shows a schematic diagram of the tissue bonding mechanism of the medical tissue adhesive of the present disclosure.

FIG. 3 shows the infrared spectrograms of four different components of the present disclosure.

FIG. 4 shows the schematic diagrams of in vitro degradation of the medical tissue adhesives in the examples and comparative example of the present disclosure.

FIG. 5 shows the schematic diagrams of the mechanical strength test results of the medical tissue adhesives in the examples and comparative example of the present disclosure.

FIG. 6 shows the antibacterial activity tests on the medical tissue adhesives in Examples 1 to 4 and Comparative example.

FIG. 7 shows the cytotoxicity tests on the medical tissue adhesives in Examples 1 to 4 and Comparative example.

FIG. 8 shows the live/dead cell staining images of L929 cells treated with the medical tissue adhesive of the comparative example and the SG/TA-0.3% medical tissue adhesive of the example.

FIG. 9 shows the schematic diagrams of the results of the stress-strain curve, tissue adhesive property, and the degradation of medical tissue adhesive in the simulated pancreatic juice tolerance test.

FIG. 10 shows the cumulative leak volume of pancreatic juice within 28 days after surgery and the content of α-amylase in pancreatic juice within 28 days after surgery.

FIG. 11 shows the stress-strain curves of the medical tissue adhesives of the example and comparative example.

FIG. 12 shows the shape change of the medical tissue adhesive of the example before and after being stretched to 80% strain.

FIG. 13 shows a schematic diagram of hematoxylin-eosin staining of an incised pancreatic duct plugged for 28 days.

### DETAILED DESCRIPTION

Various exemplary examples, features and aspects of the present disclosure will be described below in detail. The wording “exemplary” used exclusively herein means “being used as an instance or an example, or being illustrative”. Any example illustrated herein as “exemplary” is not necessarily construed as being superior to or better than other examples.

In addition, numerous specific details are given in the following specific embodiments for the purpose of better explaining the present disclosure. It should be understood by a person skilled in the art that the present disclosure may still be realized without some specific details. In some other examples, methods, means, equipments and steps that are well known to a person skilled in the art are not described in detail so as to highlight the gist of the present disclosure.

Unless otherwise stated, all of the units used in the present specification are international standard units, and the numerical values and numerical ranges appeared in the present disclosure shall all be construed to include systematic errors that are inevitable in industrial production.

In the present specification, “% (m/v)” denotes a mass percentage concentration unless otherwise specified. This mass percentage concentration refers to the mass of a component in a unit volume of solvent. The mass percentage concentration in the present specification is calculated with mL as the volume unit and with g as the mass unit. Exemplarily, if 0.003 g of a material component is added to 1 mL of a solvent, the mass percentage concentration of this material component is (0.003 g/l mL)*100%=0.3% (m/v).

In the present specification, the meaning expressed via the wording “may” encompasses the meaning of carrying out certain treatment and the meaning of not carrying out certain treatment.

In the present specification, a reference to “some specific/preferred embodiments”, “some other specific/preferred embodiments”, “embodiment(s)”, or the like means that the specific elements (such as features, structures, properties and/or characteristics) described in connection with this embodiment are included in at least one of the embodiments described herein, and may or may not exist in other embodiments. In addition, it should be appreciated that said elements may be combined into various embodiments in any suitable manner.

In the present specification, a numerical range represented by “numerical value A to numerical value B” refers to a range including the endpoint values A and B.

Additionally, in the present specification, said “water” includes any usable and feasible water such as deionized water, distilled water, ion exchange water, double distilled water, high purity water, and pure water.

In the present specification, when the wording “normal temperature” or “room temperature” is used, the temperature may be 10° C. to 40° C.

### <First Aspect>

The first aspect of the present disclosure provides a medical tissue adhesive composition, comprising a medical tissue adhesive component and a chelating agent component, wherein
the medical tissue adhesive component comprises a first component and a second component, the first component comprises four-arm polyethylene glycol amine, and the second component comprises four-arm-polyethylene glycol-succinimidyl glutarate, wherein the four-arm polyethylene glycol amine has a structural formula as represented by formula (I):

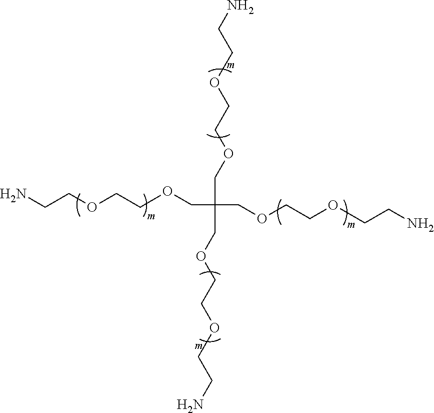

the four-arm-polyethylene glycol-succinimidyl glutarate has a structural formula as represented by formula (II):

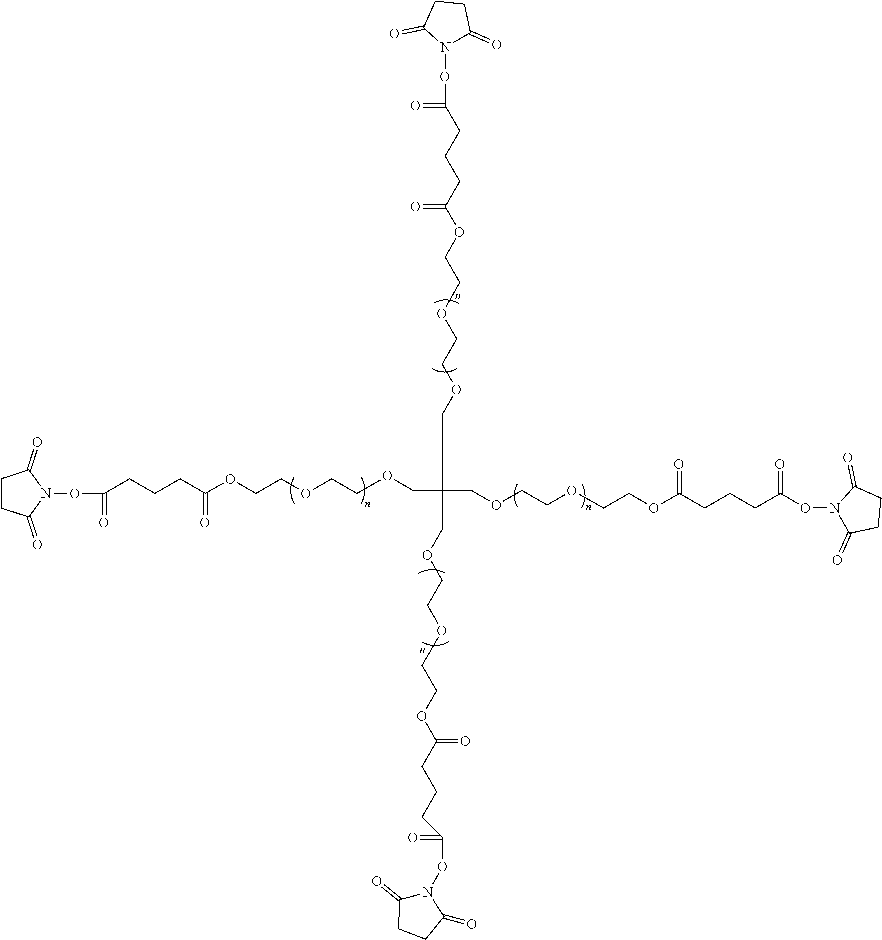

wherein m and n are natural numbers; and
the chelating agent component comprises polyphenol.

Specifically, the medical tissue adhesive component and the chelating agent component may be separated, and the medical tissue adhesive component and the chelating agent component are mixed when in use. Preferably, the first component and the second component in the medical tissue adhesive component are also separated, and the first component and the second component are mixed a few hours before use or when in use. Typically, the chelating agent component may be mixed with the first component, and then mixed with the second component to form an adhesive.

During practical application, the medical tissue adhesive component and the chelating agent component may be stored separately, and dissolved in a solvent for mixing before use or when in use. For example, it is possible to prepare a solution A comprising a first component and a chelating agent, prepare a solution B comprising a second component, and finally mix the solution A and the solution B to obtain the medical tissue adhesive. The present disclosure does not limit the storage manner for respective components in the medical tissue adhesive composition, and a person skilled in the art may select the specific storage manner as required, all of which are within the scope of the present disclosure.

In the present disclosure, four-arm-polyethylene glycol-succinimidyl glutarate and four-arm polyethylene glycol amine may be bonded to form the skeleton of the medical tissue adhesive, such that the chelating agent component is chelated with the skeleton of the medical tissue adhesive via hydrogen bond. In the present disclosure, the bonding of four-arm-polyethylene glycol-succinimidyl glutarate and four-arm polyethylene glycol amine may form a multi-arm polymer with a three-dimensional spatial structure, which is used as the skeleton of the medical tissue adhesive, and the performance of the resulting skeleton of the medical tissue adhesive is more excellent. By chelating the skeleton of the medical tissue adhesive with a chelating agent component, a medical tissue adhesive with good bacteriostatic property, mechanical properties and adhesive property, low swelling property as well as controllable gelation rate and degradation rate could be obtained. The medical tissue adhesive may instantly produce adhesive property by taking advantage of the covalent reaction of the functional groups in the skeleton of the medical tissue adhesive with the tissue, thereby satisfying the biomedical requirements for the plugging of pancreatic juice leakage.

In some specific embodiments, the four-arm-polyethylene glycol-succinimidyl glutarate has a weight-average molecular weight of 2000 to 20000 Da; and the four-arm polyethylene glycol amine has a weight-average molecular weight of 2000 to 20000 Da. By using four-arm-polyethylene glycol-succinimidyl glutarate and four-arm polyethylene glycol amine each having an appropriate weight-average molecular weight, it is more conducive to the formation of a desired skeleton of the medical tissue adhesive.

Specifically, in the present disclosure, the four-arm-polyethylene glycol-succinimidyl glutarate has a weight-average molecular weight of 2000 to 20000 Da, preferably 5000 to 15000 Da, more preferably 8000 to 12000 Da; for example, 3000 Da, 5000 Da, 8000 Da, 10000 Da, 15000 Da or 20000 Da. The four-arm polyethylene glycol amine has a molecular weight of 2000 to 20000 Da, preferably 5000 to 15000 Da, more preferably 8000 to 12000 Da; for example, 3000 Da, 4000 Da, 6000 Da, 8000 Da, 10000 Da, 12000 Da, 15000 Da, 18000 Da or 20000 Da.

The weight-average molecular weight according to the present disclosure may be determined by a method commonly used in the art, for example, the MALDI-TOF determination method.

By adjusting the weight-average molecular weights of four-arm-polyethylene glycol-succinimidyl glutarate and four-arm polyethylene glycol amine, the degradation rate of the medical tissue adhesive could be adjusted to meet the requirements for degradation and clearance at different tissue sites, and to avoid the inflammatory reaction caused by its overlong existence in the body.

The chelating agent of the present disclosure comprises polyphenol. The inventors of the present disclosure have discovered that use of the chelating agent enables the prepared medical tissue adhesive to have excellent antibacterial property. The chelating agent component of the present disclosure is released during the degradation process and is capable of producing bacteriostatic effect.

In the present disclosure, said polyphenol may include tannic acid, catechol, pyrogallol or resveratrol, or a combination of two or more selected from these components. In some preferred embodiments, the chelating agent is tannic acid. Tannic acid not only possesses excellent bacteriostatic property to greatly improve the bacteriostatic property of the medical tissue adhesive, but also has been found in the present disclosure to enable a medical tissue adhesive with significantly improved stability and mechanical properties after being chelated with the skeleton of the medical tissue adhesive formed from the two components, i.e., four-arm polyethylene glycol amine and four-arm-polyethylene glycol-succinimidyl glutarate. Moreover, the swelling ratio of the medical tissue adhesive decreases after the addition of tannic acid, indicating that tannic acid is able to be effectively embedded into the first component and the second component so as to exert convergence effect on swelling. The medical tissue adhesive could realize gelation within 20s, with rapid gelation rate and extended degradation time.

In some specific embodiments, the mass ratio of the first component to the second component is 1:(0.8 to 1.2), preferably 1:(0.9 to 1.1), more preferably 1:(0.95 to 1.05). Medical tissue adhesives with good mechanical properties could be obtained when the mass ratio of the first component to the second component is 1:(0.8 to 1.2), for example, 1:0.82, 1:0.85, 1:0.88, 1:0.9, 1:0.92, 1:0.95, 1:0.98, 1:1, 1:1.02, 1:1.05, 1:1.08, 1:1.12, 1:1.15, 1:1.18, or the like.

In the present disclosure, the content of the chelating agent component is greater than 0 based on the total mass of the four-arm-polyethylene glycol-succinimidyl glutarate. If the chelating agent component is not contained, the gelation rate and the degradation rate of the medical tissue adhesive would be too fast, thus causing adverse effects. For example, the reactants for forming the medical tissue adhesive have been fully reacted before completely covering the part where pancreatic juice leaks, which is not conducive to adequate protection of the wound surface, and secondly not conducive to provision of continuous protection during the recovery period of the wound surface. If the content of the chelating agent component is too high, the gelation rate and the degradation rate would be too slow, which does not contribute to patients' recovery. Preferably, in the present disclosure, the mass ratio of the four-arm-polyethylene glycol-succinimidyl glutarate to the chelating agent is 1:(0.003 to 0.03), preferably 1:(0.01 to 0.02), for example, 1:0.005, 1:0.008, 1:0.01, 1:0.012, 1:0.015, 1:0.018, 1:0.02, 1:0.022, 1:0.025, 1:0.028, or the like.

The components used in the present disclosure, such as four-arm-polyethylene glycol-succinimidyl glutarate, four-arm polyethylene glycol amine, and the chelating agent component, all have good biocompatibility, and exhibit excellent biocompatibility and are less likely to cause inflammatory reactions when implanted into the body compared to the potential toxic components contained in other tissue binders, such as glutaraldehyde and cyanoacrylate monomers. Typically, polyethylene glycol macromolecules having a weight-average molecular weight of 10,000 Da or less may be excreted out of the body through cell permeation and endocytosis along with metabolism and circulation despite the fact that their molecular chains would not be degraded and broken, while other tissue binders such as BioGlue and α-cyanoacrylate lose the biodegradable function of the material due to the factors such as high crosslinking density and stable intermolecular chemical force.

Furthermore, in some specific embodiments, the medical tissue adhesive composition further comprises one or a combination of two or more selected from a metal compound, a polypeptide, and an antimicrobial drug. The metal compound comprises one or a combination of two or more selected from magnesium oxide, zinc oxide, and zinc carbonate, and the antimicrobial drug comprises one or a combination of two or more selected from vancomycin, gentamicin, fusidine, and a quaternary ammonium salt.

### <Second Aspect>

The second aspect of the present disclosure provides a medical tissue adhesive, which is formed via the reaction of the first component and the second component of the medical tissue adhesive composition according to the first aspect of the present disclosure in the presence of a chelating agent component. The medical tissue adhesive of the present disclosure is a medical tissue adhesive material with high tissue adhesiveness, good biocompatibility and excellent antibacterial function.

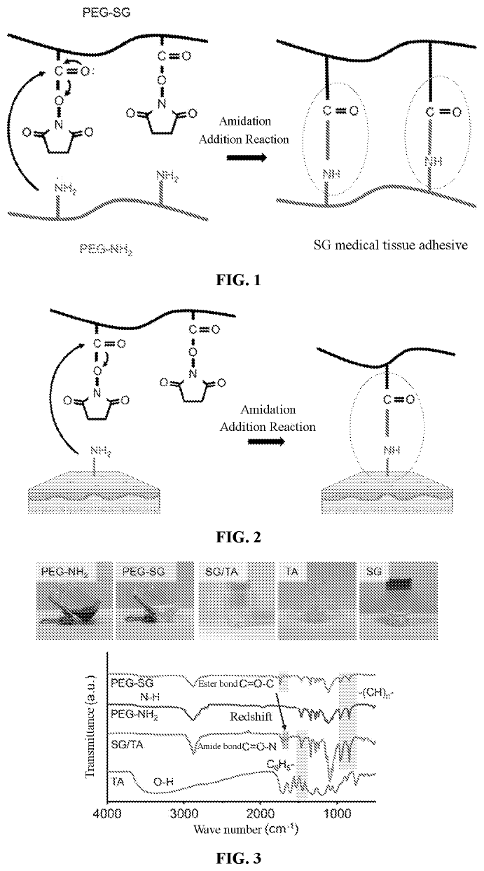

FIG. 1 shows a schematic diagram of the gelation mechanism of the medical tissue adhesive of the present disclosure. As shown in FIG. 1, the lone-pair electrons of the amino group on the four-arm polyethylene glycol amine attack the carbonyl carbon on the four-arm-polyethylene glycol-succinimidyl glutarate, thereby resulting in an amidation reaction to form a gel. FIG. 2 shows a schematic diagram of the tissue bonding mechanism of the medical tissue adhesive of the present disclosure. As can be seen from FIG. 2, the lone-pair electrons of the amino group on the biological tissue attack the carbonyl carbon in the medical tissue adhesive, thereby resulting in an amidation reaction to form tissue bonding.

The mechanism for the medical tissue adhesive composition of the present disclosure to form a gel by mixing four-arm-polyethylene glycol-succinimidyl glutarate and four-arm polyethylene glycol amine lies in that the electrophilic group N-hydroxy succinimidyl (—NHS) in the four-arm-polyethylene glycol-succinimidyl glutarate and the nucleophilic group (—NH₂) undergo an addition reaction to form a crosslinked network structure and are thus gelated. This medical tissue adhesive creates an interfacial adhesive force between the medical tissue adhesive and the tissue mainly by the interaction between unreacted N-hydroxy succinimidyl (—NHS) remained in the medical tissue adhesive and other bio-derived base materials such as the amino group (—NH₂) between tissues.

Specifically, in the present disclosure, the medical tissue adhesive has a tissue adhesive strength of 2 to 50 kPa, for example, 5 kPa, 10 kPa, 15 kPa, 20 kPa, 25 kPa, 30 kPa, 35 kPa, 40 kPa, 45 kPa, or the like. The medical tissue adhesive has a degradation cycle of 30 to 60 days, for example, 32 days, 35 days, 38 days, 40 days, 42 days, 45 days, 48 days, 50 days, 52 days, 55 days, 58 days, or the like; and the medical tissue adhesive has a bacteriostatic rate of 95% or more, for example, 96%, 97%, 98%, 99%, or the like.

### <Third Aspect>

The third aspect of the present disclosure provides a preparation method of the medical tissue adhesive according to the second aspect of the present disclosure, comprising the steps of:
preparing a solution A comprising a first component and a chelating agent;
preparing a solution B comprising a second component; and
mixing the solution A and the solution B to obtain the medical tissue adhesive.

In some embodiments, the solution A and the solution B are mixed directly on the wound surface of the tissue, so that the wound could be directly bonded and wound surface could be closed. The four-arm polyethylene glycol amine and four-arm-polyethylene glycol-succinimidyl glutarate form the medical tissue adhesive component, and it undergoes a chelation reaction with a chelating agent, such that the resulting tissue adhesive has higher strength and protects the wound surface effectively, without a decrease in viscosity due to the secretion of tissue fluids from the wound surface. With the recovery of the wound surface, the tissue adhesive gradually degrades, and the addition of the chelating agent is capable of providing bacteriostatic effect.

As for solution A, any one of the following preparation methods may be selected.

In some specific embodiments, a solution containing four-arm polyethylene glycol amine and a solution containing the chelating agent are prepared respectively, and the two solutions are mixed to obtain the solution A.

In some specific embodiments, a solution containing four-arm polyethylene glycol amine is prepared, and the chelating agent is added to the solution containing four-arm polyethylene glycol amine to obtain the solution A.

In some specific embodiments, a solution containing the chelating agent is prepared, and the four-arm polyethylene glycol amine is added to the solution containing the chelating agent to obtain the solution A.

In some specific embodiments, the four-arm polyethylene glycol amine and the chelating agent are mixed and added to a solvent to obtain the solution A.

The solution containing the four-arm polyethylene glycol amine may be obtained by dissolving the four-arm polyethylene glycol amine in a solvent; and the solution containing the chelating agent may be obtained by dissolving the chelating agent in a solvent.

The solvent is not particularly limited in the present disclosure, and it may be any feasible solvent in the art, for example, a buffer solution, water, or the like. Specifically, a buffer solution may be used to prepare the solution A containing the first component and the chelating agent. Specifically, the pH value of the buffer solution may be 7.0 to 7.4. When the pH value of the buffer solution is 7.0 to 7.4, the prepared medical tissue adhesive is approximately neutral, which is less irritating to tissues and is beneficial to the growth of cells and tissues. The buffer solution is not particularly limited in the present disclosure, and it may be some buffer solutions commonly used in the art, for example, PBS buffer solution, normal saline, or the like.

In some specific embodiments, in the solution A, the chelating agent component has a mass percentage concentration of 0.06 to 0.6% (m/v), preferably 0.1 to 0.5% (m/v), more preferably 0.2 to 0.4% (m/v), further preferably 0.3% (m/v). Exemplarily, the chelating agent in the solution A has a mass percentage concentration of 0.07% (m/v), 0.08% (m/v), 0.1% (m/v), 0.15% (m/v), 0.2% (m/v), 0.25% (m/v), 0.3% (m/v), 0.35% (m/v), 0.4% (m/v), or the like.

In the solution A, the four-arm polyethylene glycol amine has a mass percentage concentration of 10 to 25% (m/v), preferably 18 to 22% (m/v). Exemplarily, the four-arm polyethylene glycol amine in the solution A has a mass percentage concentration of 12% (m/v), 14% (m/v), 16% (m/v), 18% (m/v), 20% (m/v), 22% (m/v), 24% (m/v), or the like.

The solution B is obtained by dissolving four-arm-polyethylene glycol-succinimidyl glutarate in a solvent. The solvent may or may not be the same as that of the solution A, and may be any feasible solvent in the art, for example, a buffer solution, water, or the like. Specifically, a buffer solution may be used to prepare a solution B containing the second component. Specifically, the pH value of the buffer solution may be 7.0 to 7.4. When the pH value of the buffer solution is 7.0 to 7.4, the prepared medical tissue adhesive is approximately neutral, which is less irritating to tissues and is beneficial to the growth of cells and tissues. The buffer solution is not particularly limited in the present disclosure, and it may be buffer solutions commonly used in the art, for example, PBS buffer solution, normal saline, or the like.

In some embodiments, in the solution B, the four-arm-polyethylene glycol-succinimidyl glutarate has a mass percentage concentration of 10 to 25% (m/v), preferably 18 to 22% (m/v). Exemplarily, the four-arm-polyethylene glycol-succinimidyl glutarate in the solution B has a mass percentage concentration of 12% (m/v), 14% (m/v), 16% (m/v), 18% (m/v), 20% (m/v), 22% (m/v), 24% (m/v), or the like.

By adjusting the content of four-arm polyethylene glycol amine and the content of four-arm-polyethylene glycol-succinimidyl glutarate, the ratio between the two PEG derivatives that form the medical tissue adhesive component may be controlled, thereby improving the mechanical properties of the medical tissue adhesive component. By adjusting the content of tannic acid added, the gelation rate, degradation rate and swellability of the medical tissue adhesive may be further adjusted, thereby forming a medical tissue adhesive that is suitable for wound closure and has high mechanical strength in terms of tissue bonding, good biocompatibility, high adhesive strength, and good bacteriostatic property.

Furthermore, after the solution A and the solution B in the medical tissue adhesive composition of the present disclosure are mixed, the first component in the solution A and the second component in the solution B may undergo the Michael addition reaction, and may form a gel quickly, i.e., generating the medical tissue adhesive of the present disclosure. This medical tissue adhesive is a medical tissue adhesive material having high tissue adhesiveness, good biocompatibility, and excellent antibacterial function.

### <Fourth Aspect>

The fourth aspect of the present disclosure provides a medical tissue adhesive kit, comprising the medical tissue adhesive composition according to the first aspect of the present disclosure.

The medical tissue adhesive kit according to the present disclosure comprises a medical tissue adhesive component and a chelating agent component. The medical tissue adhesive component comprises a first component and a second component; the first component comprises four-arm polyethylene glycol amine, and the second component comprises four-arm-polyethylene glycol-succinimidyl glutarate; wherein the four-arm polyethylene glycol amine has a structural formula as represented by formula (I):

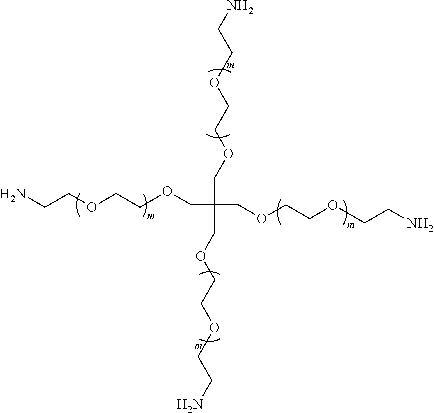

the four-arm-polyethylene glycol-succinimidyl glutarate has a structural formula as represented by formula (II):

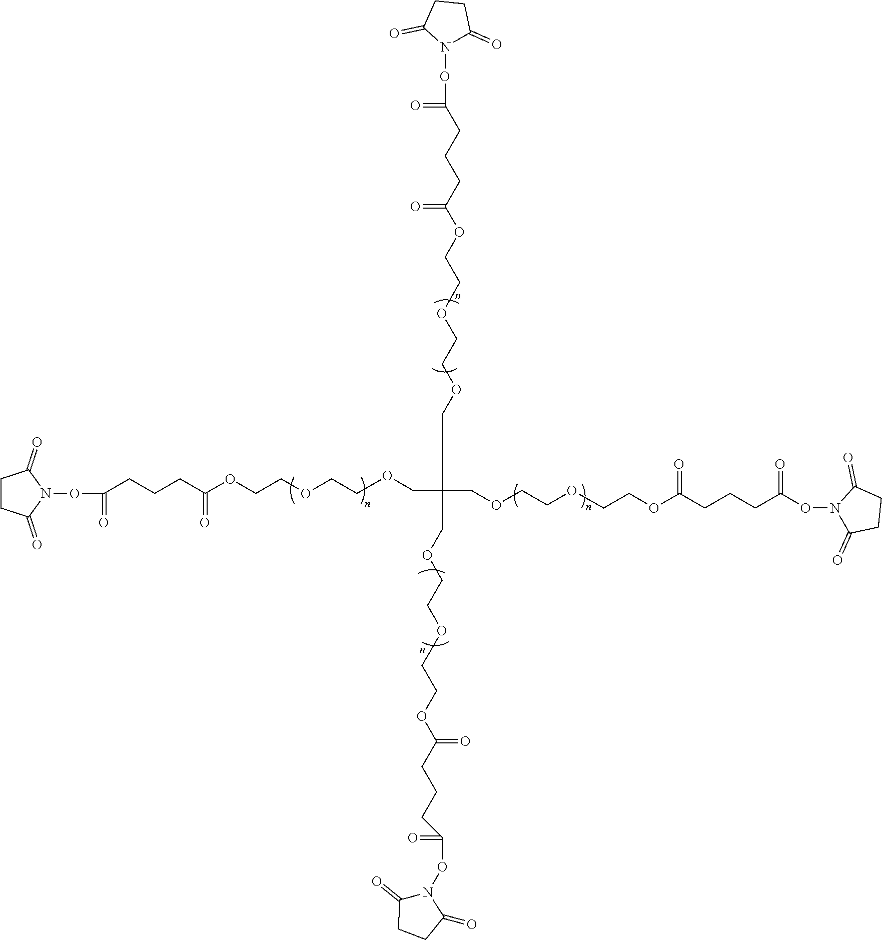

wherein m and n are natural numbers; and
the chelating agent component comprises polyphenol.

In the medical tissue adhesive kit according to the present disclosure, the medical tissue adhesive component and the chelating agent component may be stored separately, and the medical tissue adhesive component and the chelating agent component are mixed when in use. Preferably, the first component and the second component in the medical tissue adhesive component may also be stored separately, and the first component is mixed with the chelating agent component and then mixed with the second component a few hours before use or when in use.

In a specific medical tissue adhesive kit, the first component and the chelating agent component may be stored separately, and dissolved in the same solvent before use or when in use. To meet different requirements for use, the chelating agent component and the second component may be dissolved respectively in buffer solutions in advance for storage, for example, dissolved in small amounts of buffer solutions and diluted before use, or dissolved in buffer solutions for storage in accordance with the concentrations in use. When in use, the chelating agent component dissolved in the buffer solution is mixed with the first component, and then mixed with the second component dissolved in the buffer solution.

The present disclosure does not limit the storage methods and usages of respective components in the medical tissue adhesive kit, and appropriate storage methods and usages may be selected as required.

The preparation kit in the present disclosure may realize rapid preparation of a medical tissue adhesive using three components, i.e., a first component, a second component, and a chelating agent, so as to obtain a medical tissue adhesive with good bacteriostatic property, mechanical properties, adhesiveness property, and controllable gelation rate and degradation rate. Using a first component, a second component and a chelating agent to prepare a medical tissue adhesive on the wound surface of the tissue enables instant generation of adhesiveness property, which meets different biomedical requirements such as postoperative hemostasis, wound closure, and tissue bonding. Using a first component, a second component and a chelating agent to prepare a medical tissue adhesive in an environment without contact with the tissue enables the formation of a tissue engineering scaffold for tissue repair with good properties such as good mechanical properties, high biocompatibility and controllable degradation rate.

In some embodiments, the first component is a solid reagent, for example, the first component is a dry powder of four-arm polyethylene glycol amine. In some embodiments, the first component is a liquid reagent, for example, the first component is obtained by dissolving four-arm polyethylene glycol amine in a solvent. The solvent for dissolving four-arm polyethylene glycol amine may be the solvent for dissolving the first component in the third aspect.

In some embodiments, the second component is a solid reagent, for example, the second component is a dry powder of four-arm-polyethylene glycol-succinimidyl glutarate. In some embodiments, the second component is a liquid reagent, for example, the second component is obtained by dissolving four-arm-polyethylene glycol-succinimidyl glutarate in a solvent. The solvent for dissolving four-arm-polyethylene glycol-succinimidyl glutarate may be the solvent for dissolving the second component in the third aspect.

In some embodiments, the chelating agent is a solid reagent, for example, the chelating agent is a dry powder of polyphenol. In some embodiments, the chelating agent is a liquid reagent, for example, the chelating agent is obtained by dissolving polyphenol in a solvent. The solvent for dissolving the chelating agent may be the solvent for dissolving the chelating agent in the third aspect.

In some embodiments, the first component and the chelating agent in the kit may also be present in the form of a mixed reagent, for example, a mixed dry powder of four-arm polyethylene glycol amine and polyphenol, or a mixed solution in which both four-arm polyethylene glycol amine and polyphenol are dissolved.

In some embodiments, the kit further comprises instructions providing the following contents.
(1) The mass ratio of four-arm polyethylene glycol amine to four-arm-polyethylene glycol-succinimidyl glutarate in a medical tissue adhesive prepared by the kit is 1:(0.8 to 1.2), preferably 1:(0.9 to 1.1), more preferably 1:(0.95 to 1.05).
(2) The mass ratio of four-arm polyethylene glycol amine to the chelating agent in a medical tissue adhesive prepared by the kit is 1:(0.003 to 0.03), preferably 1:(0.01 to 0.02).

In some embodiments, the kit further comprises instructions, and the instructions in the kit provide the following contents:
(1) preparing a solution A containing four-arm polyethylene glycol amine and a chelating agent from the first component and the chelating agent;
(2) preparing a solution B containing four-arm-polyethylene glycol-succinimidyl glutarate from the second component; and
(3) mixing the solution A and the solution B to obtain the medical tissue adhesive.

### <Fifth Aspect>

The fifth aspect of the present disclosure provides the use of the medical tissue adhesive according to the second aspect of the present disclosure or a medical tissue adhesive prepared and obtained by the preparation method for the medical tissue adhesive according to the third aspect of the present disclosure in the preparation of a plugging article for pancreatic juice leakage.

Furthermore, the medical tissue adhesive of the present disclosure may further be used to prepare hemostatic aids, articles for preventing pulmonary air leakage or cerebrospinal fluid leakage, anti-adhesion articles and the like, for example, applied to plugging of needle holes caused by suture in arteriovenous reconstruction, plugging of needle holes caused by dural suturing, bonding and plugging in dural reconstruction, prevention of pulmonary air leakage after pneumonectomy, anti-adhesion of spinal dura, etc. The medical tissue adhesive of the present disclosure may achieve effective bonding to tissues to quickly stop bleeding, effectively close wounds and promote the healing of wound tissues, and has potentially huge application scenarios in many fields.

### EXAMPLES

The embodiments of the present disclosure will be described in detail below in conjunction with examples. However, a person skilled in the art would understand that the following examples are merely used for explaining the present disclosure and should not be deemed to restrict the scope of the present disclosure. Where the specific conditions are not indicated in the examples, conventional conditions or the conditions recommended by the manufacturers shall be followed. Reagents or instruments, whose manufacturers are not specified, are all conventional products that are commercially available.

### Example 1

In this example, tannic acid, 4arm-PEG-NH₂ and 4arm-PEG-SG were used as raw materials to prepare a medical hydrogel. The 4arm-PEG-NH₂ had a structure represented by formula (I) and had a weight-average molecular weight of 10000 Da; and the 4arm-PEG-SG had a structure represented by formula (II), and 4arm-PEG-SG had a weight-average molecular weight of 10000 Da.

The specific preparation steps of the medical hydrogel were as follows.

(1) 0.001 g of tannic acid and 0.2 g of 4arm-PEG-NH₂ were taken, mixed, and then dissolved in 1 mL of PBS buffer solution with a pH of 7.2 to 7.4 to obtain a solution A. In the solution A, the mass percentage concentration of tannic acid was 0.10% (m/v), and the mass percentage concentration of 4arm-PEG-NH₂ was 20% (m/v). The solution A was set aside for use.

(2) 0.2 g of 4arm-PEG-SG was taken and dissolved in 1 mL of PBS buffer solution with a pH value of 7.2 to 7.4, and after complete dissolution, a solution B containing 20% (m/v) 4arm-PEG-SG was obtained and set aside for use.

(3) The above solution A and solution B were added to a dual syringe and injected concurrently into the site to be plugged to form an adhesive and plug the pancreatic duct, and the adhesive was recorded as SG/TA-0.1%.

### Example 2

In this example, tannic acid, 4arm-PEG-NH₂ and 4arm-PEG-SG were used as raw materials to prepare a medical hydrogel. The 4arm-PEG-NH₂ had a structure represented by formula (I) and had a weight-average molecular weight of 5000 Da; and the 4arm-PEG-SG had a structure represented by formula (II) and had a weight-average molecular weight of 5000 Da.

The specific preparation steps of the medical hydrogel were as follows.

(1) 0.003 g of tannic acid was taken and dissolved in 1 mL of PBS buffer solution with a pH value of 7.2 to 7.4.

(2) 0.1 g of 4arm-PEG-NH₂ was taken and dissolved in the solution of tannic acid.

(3) The above two solutions were mixed to obtain a solution A. In the solution A, the mass percentage concentration of tannic acid was 0.3% (m/v), and the mass percentage concentration of 4arm-PEG-NH₂ was 10% (m/v). The solution A was set aside for use.

(4) 0.1 g of 4arm-PEG-SG was taken and dissolved in 1 mL of PBS buffer solution with a pH of 7.2 to 7.4, and after complete dissolution, a solution B containing 10% (m/v) 4arm-PEG-SG was obtained and set aside for use.

(5) Solution A and solution B were added to syringes and injected concurrently into the site to be plugged to form an adhesive and plug the pancreatic duct, and the adhesive was recorded as SG/TA-0.3%.

### Example 3

In this example, tannic acid, 4arm-PEG-NH₂ and 4arm-PEG-SG were used as raw materials to prepare a medical hydrogel. The 4arm-PEG-NH₂ had a structure represented by formula (I) and had a weight-average molecular weight of 20000 Da; and the 4arm-PEG-SG had a structure represented by formula (II) and had a weight-average molecular weight of 20000 Da.

The specific preparation steps of the medical hydrogel were as follows.

(1) 0.25 g of 4arm-PEG-NH₂ was taken and dissolved in 1 mL of PBS buffer solution with a pH of 7.2 to 7.4, and after 4arm-PEG-NH₂ was completely dissolved, a solution was obtained, in which the mass percentage concentration of 4arm-PEG-NH₂ was 25% (m/v).

(2) 0.005 g of tannic acid was taken and dissolved in the solution formulated in step (1), and after dissolution, the mass percentage concentration of tannic acid was 0.5% (m/v). The solution A was obtained and set aside for use.

(3) 0.25 g of 4arm-PEG-SG was taken and dissolved in 1 mL of PBS buffer solution with a pH value of 7.2 to 7.4, and after complete dissolution, 25% (m/v) solution B was obtained and set aside for use.

(4) The above solution A and solution B were added to a dual syringe and injected concurrently into the site to be plugged to form an adhesive and plug the pancreatic duct, and the adhesive was recorded as SG/TA-0.5%.

### Example 4

In this example, tannic acid, 4arm-PEG-NH₂ and 4arm-PEG-SG were used as raw materials to prepare a medical hydrogel. The 4arm-PEG-NH₂ had a structure represented by formula (I) and had a weight-average molecular weight of 15000 Da; and the 4arm-PEG-SG had a structure represented by formula (II) and had a weight-average molecular weight of 10000 Da.

The specific preparation steps of the medical hydrogel were as follows.

(1) 0.1 g of tannic acid and 2 g of 4arm-PEG-NH₂ were taken and dissolved in 10 mL of PBS buffer solution with a pH of 7.2 to 7.4 to obtain a solution A after tannic acid and 4arm-PEG-NH₂ were completely dissolved. In the solution A, the mass percentage concentration of tannic acid was 1.0% (m/v), and the mass percentage concentration of 4arm-PEG-NH₂ was 20% (m/v). The solution A was set aside for use.

(2) 2 g of 4arm-PEG-SG was taken and dissolved in 10 mL of PBS buffer solution with a pH of 7.2 to 7.4, and after complete dissolution, a solution B of 4arm-PEG-SG was obtained, in which the mass percentage concentration of 4arm-PEG-SG was 20% (m/v). The solution B was set aside for use.

(3) The above solution A and solution B were added to a dual syringe and injected concurrently into the site to be plugged to form an adhesive and plug the pancreatic duct, and the adhesive was recorded as SG/TA-1.0%.

### Comparative Example 1

In this comparative example, 4arm-PEG-NH₂ and 4arm-PEG-SG were used as raw materials to prepare a medical hydrogel. The 4arm-PEG-NH₂ had a structure represented by formula (I) and had a weight-average molecular weight of 10000 Da; and the 4arm-PEG-SG had a structure represented by formula (II) and had a weight-average molecular weight of 10000 Da.

The specific preparation steps of the medical hydrogel were as follows.

(1) 0.2 g of 4arm-PEG-NH₂ was taken and dissolved in 1 mL of PBS buffer solution with a pH of 7.2 to 7.4, and after dissolution, a 4arm-PEG-NH₂ solution having a mass percentage concentration of 20% (m/v) was obtained.

(2) 0.2 g of 4arm-PEG-SG was taken and dissolved in 1 mL of PBS buffer solution with a pH of 7.2 to 7.4, and after complete dissolution, a 4arm-PEG-SG solution having a mass percentage concentration of 20% (m/v) was obtained and set aside for use.

(3) The above 4arm-PEG-SG solution and 4arm-PEG-NH₂ solution were added to a dual syringe and injected concurrently into molds or wound surfaces of tissues to obtain respective reference samples, which were recorded as SG.

### Performance Test

The medical tissue adhesives of Examples 1 to 4 and Comparative Example 1 were subjected to the following tests.

### 1. Infrared Spectroscopic Experiment

Four-arm-polyethylene glycol-succinimidyl glutarate (denoted by PEG-SG), four-arm polyethylene glycol amine (denoted by PEG-NH₂), the medical tissue adhesive (denoted by SG/TA), and a chelating agent-free medical tissue adhesive (denoted by SG) were subjected to the infrared spectroscopic experiment. The results were shown in FIG. 3.

For ease of observation, the solution of four-arm polyethylene glycol amine (PEG-NH₂) in PBS was added with coomassie brilliant blue and appeared blue; the solution of four-arm-polyethylene glycol-succinimidyl glutarate (denoted by PEG-SG) in PBS was colorless; and tannic acid (TA) was brownish-yellow powder. When these three components were prepared into the medical tissue adhesives of the examples, gels were formed. The redshift from the ester bond to the amide bond could be observed in the infrared spectrum, indicating that gelation occurred.

### 2. In Vitro Degradation Experiment

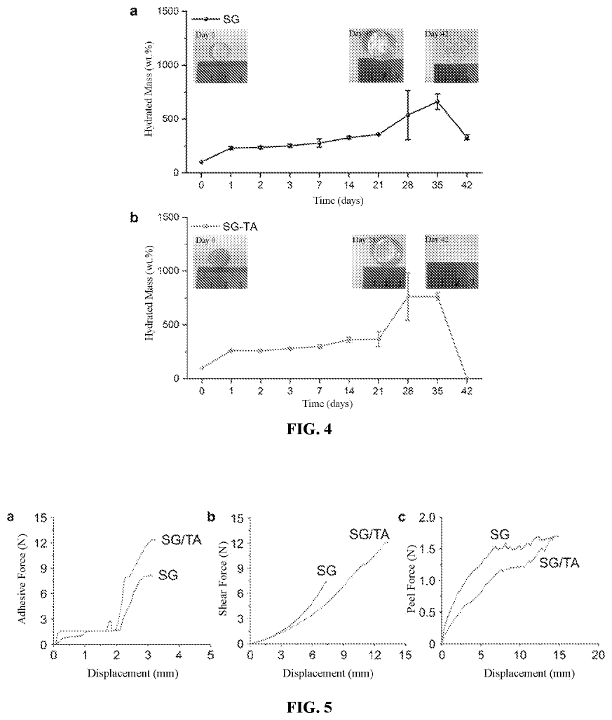

A cylindrical medical tissue adhesive (diameter: 1 cm) with a size of 1 cm³ was weighed and the initial mass was recorded as W₀. Thereafter, the cylindrical medical tissue adhesive was soaked in 10 mL of simulated pancreatic juice, placed in a constant temperature shaker at 37° C., 60 rpm, and taken out and weighed again after every specified time, and the mass was recorded as Wₜ. Hydrated mass wt. %=Wₜ/W₀×100%. Samples were set in triplicate and averaged. The test results were shown in FIG. 4.

As could be seen from FIG. 4, during the soaking in a neutral phosphate buffer solution (PBS), the medical tissue adhesive first underwent a swelling process, the PEG component regained water solubility as the crosslinked chemical bonds were further broken, and the physical structure of the medical tissue adhesive collapsed and degraded.

### 3. Mechanical Strength Test

### 3.1. Strength Test of Adhesive Force

The adhesive strength test was carried out by referring to ASTM F2258. An electronic universal testing machine was required to be equipped for the experimental operations. The purchased fresh dorsal pigskin was simply rinsed and soaked with lipase three times to remove the fat on the surface (the ratio of pigskin to lipase was 100 g of pigskin to 500 mL of lipase, the lipase concentration was 2 wt. %, and the ultrasonic treatment at a power of 240 W lasted for 30 min each time and was repeated three times), and then cut into squares in a size of 2 cm×2 cm. The smoother surfaces of two pieces of pigskin were firmly stuck with 502 super glue to two molds used for the adhesive strength test respectively, and were slightly compacted and fixed for 1 min. Thereafter, 100 μL of the medical tissue adhesives of the examples and comparative example were sprayed evenly on the surface of pigskin, and two pieces of pigskin were bonded quickly and fixed at room temperature for 60 min for subsequent test. The spline was stretched at a tensile rate of 1 mm/min at room temperature until two pieces of pigskin were broken. The force-displacement data were recorded. A force-displacement curve was plotted and analyzed. The maximum adhesive force (Fmax) was recorded. The results were shown in FIG. 5.

### 3.2. Strength Test of Shear Force

The shear strength test was carried out by referring to ASTM F2255. The fresh dorsal pigskin was defatted and then cut into a size of 5 cm×2.5 cm. Thereafter, 100 μL of the medical tissue adhesives of the examples and comparative example were applied evenly on one end of the pigskin with a coating area of 2.5 cm×1 cm, and the two ends of the pigskin were quickly bonded in opposite directions (the bonding area was the area coated with tissue adhesive), and fixed at room temperature for 30 min. The pigskin was stretched at a tensile rate of 5 mm/min at room temperature until breakage occurred. The force-displacement data were recorded. A force-displacement curve was plotted and analyzed. The maximum shear force (Fmax) was recorded. The results were shown in FIG. 5.

### 3.3. Strength Test of Peel Force

The peel strength test was carried out by referring to ATSM F2256. The fresh dorsal pigskin was defatted and then cut into a size of 5 cm×2.5 cm. Thereafter, 100 μL of the medical tissue adhesives of the examples and comparative example were applied evenly on one end of the pigskin with a coating area of 2.5 cm×1 cm, and the two ends of the pigskin were quickly bonded in the same direction (the bonding area was the area coated with tissue adhesive), and fixed at room temperature for 30 min. The pigskin was stretched at a tensile rate of 5 mm/min at room temperature until breakage occurred. The force-displacement data were recorded. A force-displacement curve was plotted and analyzed. The maximum peel force (Fmax) was recorded. The results were shown in FIG. 5.

As could be seen from FIG. 5, when the medical tissue adhesive contained four-arm polyethylene glycol amine, both the adhesive force and the shear force were greatly improved, and the peel force was also higher. Therefore, in the present disclosure, the crosslinking density of the medical tissue adhesive was improved by using four-arm polyethylene glycol amine, and the tissue adhesive property of the material was improved by four-arm polyethylene glycol amine as a reinforcing matrix.

### 4. Antibacterial Activity Test

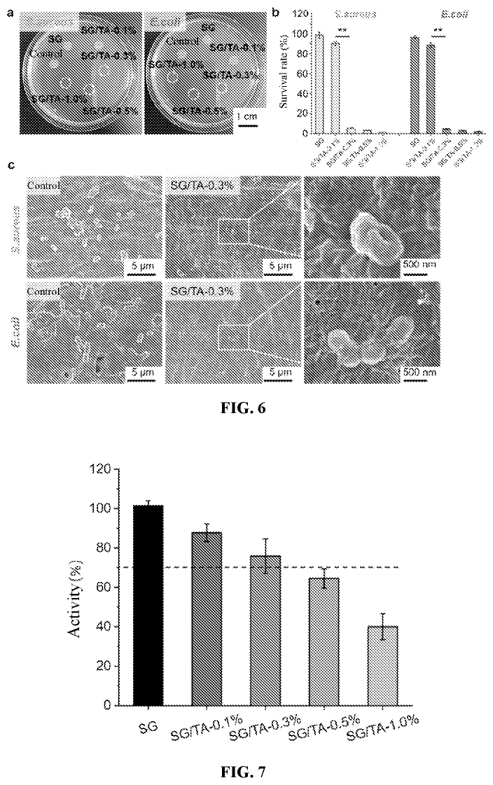

The medical tissue adhesives of Examples 1 to 4 and the Comparative Example were taken and used for the experiment, in which the bacterial colonies used were S. aureus and E. coli. 1 cm³ cylindrical medical tissue adhesives with different concentrations of tannic acid (TA) were cultured in 10 mL of culture medium containing bacteria (105 CFU/mL) at 37° C. for 24 h. The experiment was divided into three steps. 1) 10 μL of the supernatant was aspirated, dropped on a sterile agar culture plate, and continued to be cultured at 37° C. for 24 h to observe the generation of bacterial colonies; 2) 100 μL of the supernatant was aspirated and added to an Elisa plate to test the optical density (OD) value at 600 nm, survival rate (%)=(ODₛₐₘₚₗₑ−OD_{Blank})/(OD_{Bacteria}−OD_{Blank})×100%, where ODₛₐₘₚₗₑ indicated the value obtained from each of the culture media in which different tissue adhesives and bacteria were co-cultured, OD_{Bacteria} indicated the value of bacteria in the normal bacterial culture medium, and OD_{Blank} indicated the value obtained from the bacteria-free bacterial culture medium (i.e., the control group); samples were set in quadruplicate; 3) 10 μL of the supernatant was aspirated and added to 10 mL of bacteria-free bacterial culture medium containing 1 cm² of a commercial biological patch, and the resultant was cultured continuously at 37° C. for 24 h. Afterwards, the biological patch was taken out and rinsed carefully with sterile PBS three times. The sample was fixed with 4% paraformaldehyde solution for 4 h, dried, and then placed under SEM to observe the bacteria on the surface of the biological patch. The results were shown in FIG. 6.

FIG. 6 showed the antibacterial activity test on the medical tissue adhesives. In FIG. 6, a showed the generation of the bacterial colonies after the co-culture of the extracts of the medical tissue adhesives with different addition amounts of tannic acid (TA) and bacteria; b in FIG. 6 showed the optical quantitative values obtained after the co-culture of the medical tissue adhesives with different addition amounts of tannic acid (TA) and bacteria; and c in FIG. 6 showed the scanning electron micrographs illustrating the generation of bacteria on the surfaces of patches with or without the incorporation of SG/TA-0.3%.

As could be seen from FIG. 6, the antibacterial activity of the medical tissue adhesive of the present disclosure was dose-dependently relevant to the addition amount of tannic acid (TA). In the present disclosure, when the content of the chelating agent was 0.3% or more, the broad-spectrum antibacterial activity occurred, the bacteriostatic rate could reach 95%, and the biofilm on the surface of bacteria was destroyed with the release of TA.

### 5. Cytotoxicity Test

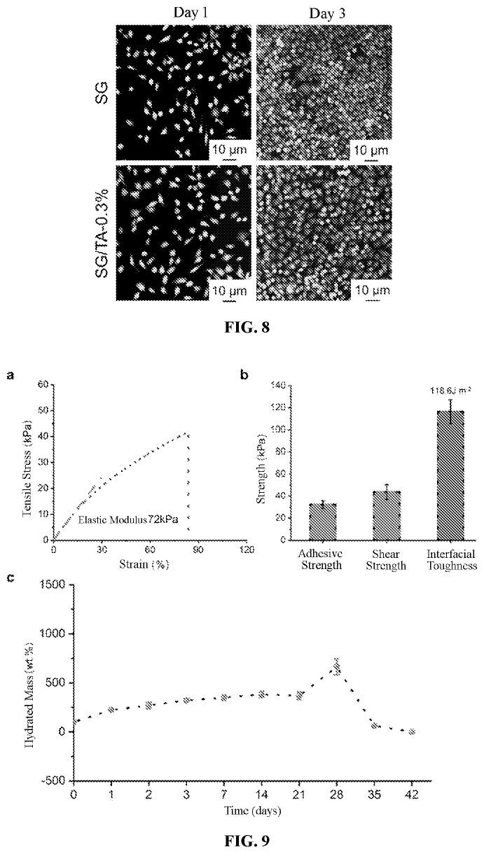

The medical tissue adhesives of Examples 1 to 4 and the Comparative Example were taken and used for the experiment. Referring to GB16886, L929 fibroblasts were selected and co-cultured for 24 h with the extracts of SG/TA or SG with different addition amounts of tannic acid (TA), and then the cells were incubated with CCK-8. The results of the cell viability were shown in FIG. 7.

As could be seen from FIG. 7, the cell viability was negatively correlated with the addition amount of tannic acid (TA), but good cell compatibility could still be achieved by controlling an appropriate dose, e.g., SG/TA-0.3%.

Further, the medical tissue adhesive of the Comparative Example and the SG/TA-0.3% medical tissue adhesive of the Example were subjected to the live/dead cell staining of L929 cells. The results were shown in FIG. 8.

As could be seen from FIG. 8, by controlling an appropriate addition amount of tannic acid (TA), the medical tissue adhesive had good cell compatibility, and the cells were able to proliferate sustainably in the extract of the medical tissue adhesive with good spreading state.

### 7. Test on Tolerance for Simulated Pancreatic Juice

Simulated pancreatic juice was formulated. 0.7 g of KH₂PO₄ was dissolved in 25 mL of deionized water until it was completely dissolved, 19 mL of 0.2 mol/L NaOH solution and 40 mL of deionized water were added and 1.0 g of trypsin was added. 0.2 mol/L of NaOH solution was added to adjust the pH to 7.5. Deionized water was added to the volume of 100 mL. The simulated pancreatic juice was formulated right before use. In accordance with the contents described in Example 2, PEG-SG, PEG-NH₂ and TA were dissolved in the simulated pancreatic juice, and then the resultant was injected via a dual syringe till the completion of customized molding, thereby obtaining a medical tissue adhesive. Afterwards, (a) the stress-strain curve (in terms of tensile) of the medical tissue adhesive was tested at room temperature; (b) the tissue adhesive property of the medical tissue adhesive gelated in the simulated pancreatic juice was tested; and (c) the degradation of the medical tissue adhesive soaked in the simulated pancreatic juice was tested. The results were shown in FIG. 9.

As could be seen from FIG. 9, the elastic modulus of the medical tissue adhesive in the simulated pancreatic juice was not much changed as compared to that in PBS (tested under normal conditions) (the elastic modulus in the simulated pancreatic juice was 72 kPa, and the elastic modulus tested under normal conditions was 76 kPa, so there was no significant difference) and the medical tissue adhesive still possessed good tissue adhesiveness and its degradation behaviors were similar to those in PBS. This indicated that the prepared tissue adhesive possessed good tolerance for pancreatic juice and was usable for plugging pancreatic leakage, while other tissue sealing materials, such as cyanoacrylate, were difficult to polymerize and bond to the wet tissue surface in the wet environment caused by pancreatic leakage, or other tissue sealing materials did not have the clinical indication of plugging pancreatic leakage, thus being difficult to be used for plugging pancreatic leakage.

### 8. Pancreatic Resection Experiment on Bama Pig

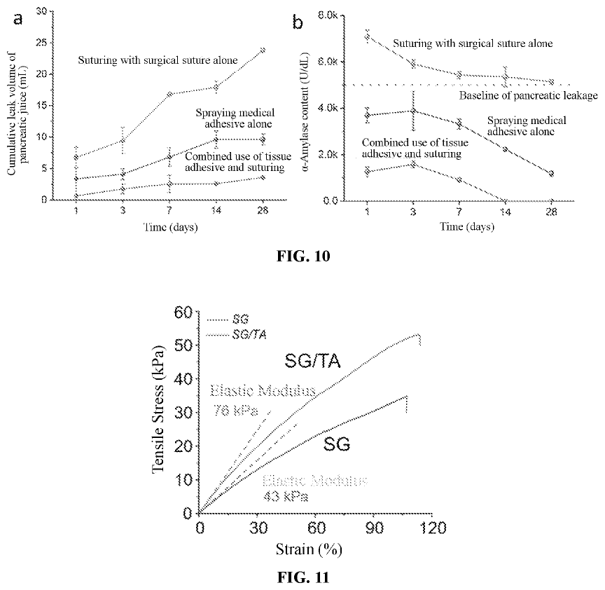

As shown in FIG. 10, the pancreatic resection experiment was carried out on Bama pig and the specific steps were as follows. (a) The pancreatic tissue was found firstly during the surgery; (b) the pancreas was incised with a surgical electrocoagulation knife to expose the pancreatic duct; (c) the cross section was sprayed only with the medical tissue adhesive, which was labeled as Group C; (d) the cross section was sutured only with surgical sutures, which was labeled as Group N; and (e) the cross section was closed with the medical tissue adhesive in combination with the suture by surgical sutures, which was labeled as Group E. Then the cumulative leak volume of pancreatic juice within 28 days after surgery and the content of α-amylase in the pancreatic juice within 28 days after surgery were detected. The results were shown in FIG. 10, where a in FIG. 10 showed the cumulative leak volume of pancreatic juice within 28 days after surgery and b in FIG. 10 showed the content of α-amylase in the pancreatic juice within 28 days after surgery.

As could be seen from FIG. 10, use of the medical tissue adhesive achieved superior efficiency in plugging pancreatic leakage as compared to suturing with surgical sutures; in case of suturing with the surgical sutures alone, pancreatic leakage still occurred, while their combination achieved the optimum therapeutic effect in clinical practice.

Additionally, FIG. 13 showed a schematic diagram of hematoxylin-eosin staining of an incised pancreatic duct plugged for 28 days. As could be seen from FIG. 13, suturing with surgical sutures alone might puncture capillaries and caused bleeding, while the combination of medical tissue adhesive and surgical suture could significantly improve the efficiency of pluging pancreatic leakage and promote tissue healing.

### 9. Stress-Strain Test

The medical tissue adhesive was injected into a customized dumbbell-shaped polytetrafluoroethylene mold (effective tensile length: 2 cm, width: 0.5 cm, depth: 0.5 cm) to make it fully filled without obvious bubbles in the medical tissue adhesive. The test was continued after the formation of the medical tissue adhesive was completed (about 10 min). The spline was taken out carefully for the detection of elastic modulus. Both ends of the medical adhesive were fixed to ensure the effective length of 2 cm prior to the test. Parameters for test sample were set (distance: 2 cm, length: 2 cm, thickness: 0.5 cm, width: 0.5 cm). All irrelevant values (including force, displacement, time, etc.) were cleared prior to the test. The spline was stretched at a tensile rate of 5 mm/min at room temperature until the spline was broken. The stress-strain data were recorded. The stress-strain curve was plotted and analyzed. The slope in the elastic deformation stage of the material in the diagram was the elastic modulus of the medical tissue adhesive. The results were shown in FIG. 11.

As could be seen from FIG. 11, the addition of tannic acid (TA) could significantly improve the mechanical strength of the medical tissue adhesive from the initial elastic modulus of 43 kPa to 76 kPa.

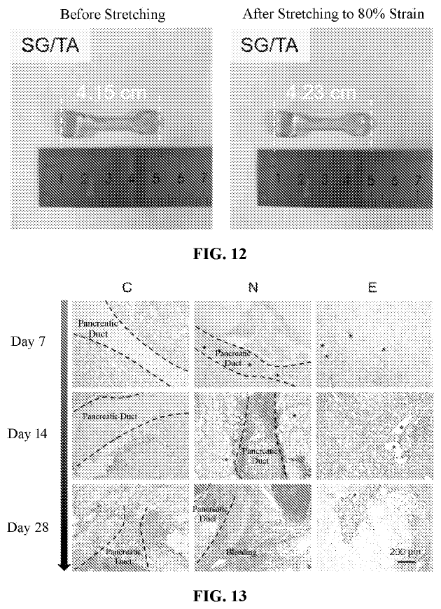

In addition, the spline was stretched to 80% strain according to the above test method, and the shape change before and after being stretched to 80% strain was observed. The results were shown in FIG. 12.

FIG. 12 showed the shape change of the medical tissue adhesive of the example before and after Bing stretched to 80% strain. As could be seen from FIG. 12, the addition of tannic acid (TA) also endowed the medical tissue adhesive with certain rebound resilience.

The above-described examples of the present disclosure are merely examples given for the purpose of clearly illustrating the present disclosure and are not intended to limit the embodiments of the present disclosure. For those of ordinary skill in the art, other variations or alterations in different forms may be further made on the basis of the above illustration. It is unnecessary and impossible to exhaustively list all embodiments here. Any modifications, equivalent alternatives, improvements and the like made within the spirits and principles of the present disclosure shall be encompassed within the protection scope of the claims of the present disclosure.

1. A medical tissue adhesive composition for preparing a plugging article for pancreatic juice leakage, comprising a medical tissue adhesive component and a chelating agent component, wherein
the medical tissue adhesive component comprises a first component and a second component, the first component comprises four-arm polyethylene glycol amine, and the second component comprises four-arm-polyethylene glycol-succinimidyl glutarate, wherein
the four-arm polyethylene glycol amine has a structural formula as represented by formula (I): 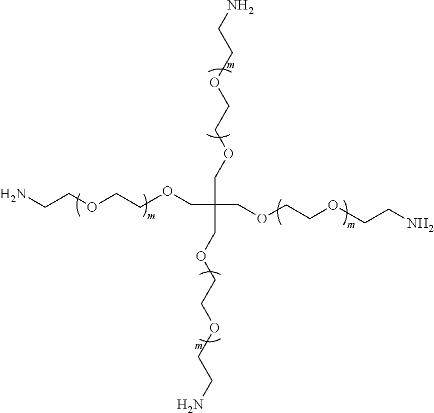
the four-arm-polyethylene glycol-succinimidyl glutarate has a structural formula as represented by formula (II): 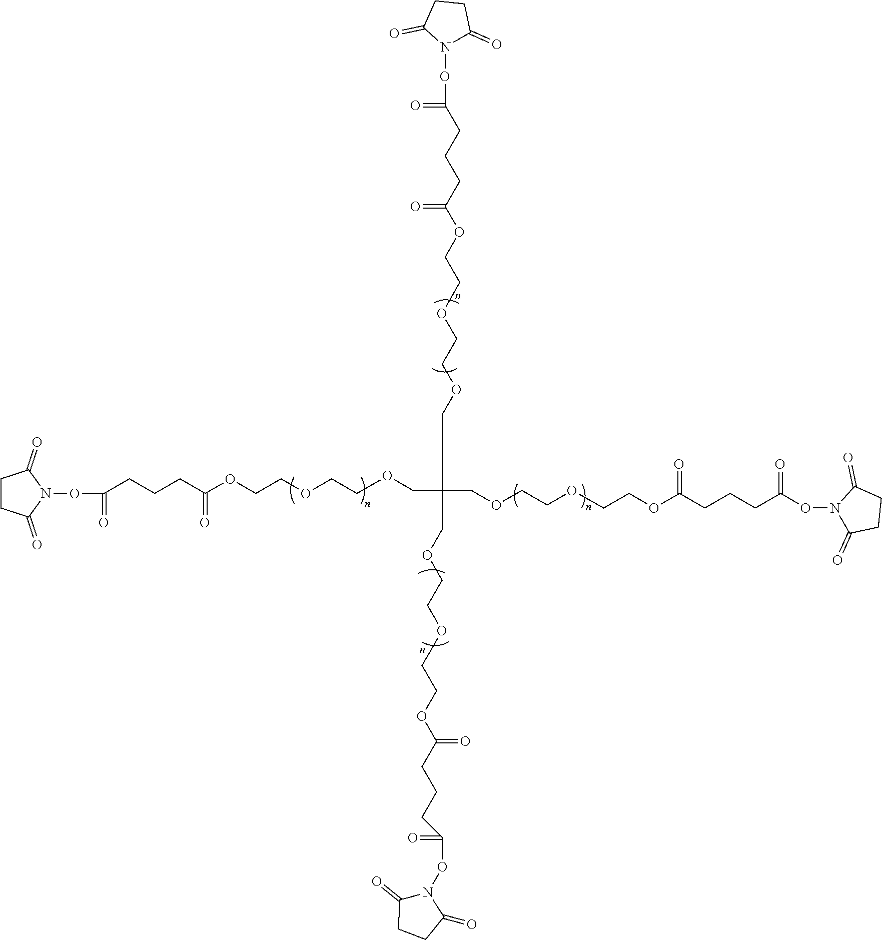
wherein m and n are natural numbers; and
the chelating agent component comprises polyphenol.

2. The medical tissue adhesive composition according to claim 1, wherein the four-arm-polyethylene glycol-succinimidyl glutarate has a weight-average molecular weight of 2000 to 20000 Da; and the four-arm polyethylene glycol amine has a weight-average molecular weight of 2000 to 20000 Da.

3. The medical tissue adhesive composition according to claim 1, wherein the polyphenol comprises tannic acid, catechol, pyrogallol or resveratrol, or a combination of two or more selected from these components.

4. The medical tissue adhesive composition according to claim 1, wherein a mass ratio of the four-arm-polyethylene glycol-succinimidyl glutarate to the chelating agent is 1:(0.003 to 0.03); and/or
a mass ratio of the first component to the second component is 1:(0.8 to 1.2).

5. The medical tissue adhesive composition according to claim 1, wherein the medical tissue adhesive composition further comprises one or a combination of two or more selected from a metal compound, a polypeptide, and an antimicrobial drug; the metal compound comprises one or a combination of two or more selected from magnesium oxide, zinc oxide, and zinc carbonate, and the antimicrobial drug comprises one or a combination of two or more selected from vancomycin, gentamicin, fusidine, and a quaternary ammonium salt.

6. A medical tissue adhesive for preparing a plugging article for pancreatic juice leakage, wherein the medical tissue adhesive is formed via the reaction of the first component and the second component of the medical tissue adhesive composition according to claim 1 in the presence of a chelating agent component.

7. The medical tissue adhesive according to claim 6, wherein the medical tissue adhesive has at least one of the following characteristics:
the medical tissue adhesive having a tissue adhesive strength of 2 to 50 kPa;
the medical tissue adhesive having a degradation cycle of 30 to 60 days; and
the medical tissue adhesive having a bacteriostatic rate of 95% or more.

8. A preparation method of the medical tissue adhesive according to claim 6, comprising the steps of:
preparing a solution A comprising a first component and a chelating agent;
preparing a solution B comprising a second component; and
mixing the solution A and the solution B to obtain the medical tissue adhesive.

9. The preparation method according to claim 8, wherein the step of preparing the solution A comprising four-arm polyethylene glycol amine and the chelating agent comprises:
preparing a solution containing four-arm polyethylene glycol amine and a solution containing the chelating agent respectively, and mixing two solutions to obtain the solution A; or
preparing a solution containing four-arm polyethylene glycol amine, and adding the chelating agent to the solution containing the four-arm polyethylene glycol amine to obtain the solution A; or
preparing a solution containing the chelating agent, and adding the four-arm polyethylene glycol amine to the solution containing the chelating agent to obtain the solution A; or
mixing the four-arm polyethylene glycol amine and the chelating agent, and adding the mixture to a solvent to obtain the solution A.

10. The preparation method according to claim 8, wherein in the solution A, the chelating agent component has a mass percentage concentration of 0.06 to 0.6% (m/v); and/or
in the solution A, the four-arm polyethylene glycol amine has a mass percentage concentration of 10 to 25% (m/v).

11. The preparation method according to claim 8, wherein in the solution B, the four-arm-polyethylene glycol-succinimidyl glutarate has a mass percentage concentration of 10 to 25% (m/v).

12. A medical tissue adhesive kit, comprising the medical tissue adhesive composition according to claim 1.

13. A method for pancreatic juice leakage, comprising administering the medical tissue adhesive according to claim 6.

14. The medical tissue adhesive composition according to claim 1, wherein the four-arm-polyethylene glycol-succinimidyl glutarate has a weight-average molecular weight of 5000 to 15000 Da; and the four-arm polyethylene glycol amine has a weight-average molecular weight of 5000 to 15000 Da.

15. The medical tissue adhesive composition according to claim 1, wherein the four-arm-polyethylene glycol-succinimidyl glutarate has a weight-average molecular weight of 8000 to 12000 Da; and the four-arm polyethylene glycol amine has a weight-average molecular weight of 8000 to 12000 Da.

16. The medical tissue adhesive composition according to claim 1, wherein a mass ratio of the four-arm-polyethylene glycol-succinimidyl glutarate to the chelating agent is 1:(0.01 to 0.02); and/or
a mass ratio of the first component to the second component is 1:(0.9 to 1.1).

17. The medical tissue adhesive composition according to claim 4, wherein a mass ratio of the first component to the second component is 1:(0.95 to 1.05).

18. The preparation method according to claim 8, wherein in the solution A, the chelating agent component has a mass percentage concentration of 0.1 to 0.5% (m/v); and/or
in the solution A, the four-arm polyethylene glycol amine has a mass percentage concentration of 18 to 22% (m/v).

19. The preparation method according to claim 8, wherein in the solution B, the four-arm-polyethylene glycol-succinimidyl glutarate has a mass percentage concentration of 18 to 22% (m/v).

20. The medical tissue adhesive kit according to claim 12, wherein the medical tissue adhesive component and the chelating agent component of the medical tissue adhesive composition being stored separately; or the first component and the second component of the medical tissue adhesive composition being stored separately.
